(19) United States
(12) Patent Application Publication
(10) Pub. No.: US 2009/0171138 A1
(43) Pub. Date: Jul. 2, 2009

(54) Ultrasonic device for fertility control and management and navigation

(76) Inventor: Uri Eli, Kiryat Ono (IL)

Correspondence Address: DR. D. GRAESER LTD., 9003 FLORIN WAY, UPPER MARLBORO, MD, 20772, US

(21) Appl. No.: 12/318,309

(22) Filed: Dec. 24, 2008

Related U.S. Application Data
(60) Provisional application No. 61/006,149, filed on Dec. 26, 2007.

(51) Int. Cl. A61F 6/02; C12N 13/00; A61B 17/425; A61F 6/06

(52) U.S. Cl. 600/33; 435/173.9; 128/842; 128/830

(57) ABSTRACT

A device for fertility control and management through the application of acoustic energy, preferably ultrasound. According to various embodiments of the present invention, such fertility management and control may optionally be applied for reducing or enhancing fertility and/or otherwise controlling one or more aspects of fertility and conception, including but not limited to contraceptive use, improving the ability to conceive, and/or for separation of X and Y sperm.

This Application claims priority from U.S. Provisional Application No. 61/006,149, filed on Dec. 26, 2007, which is hereby incorporated by reference as if fully set forth herein.

### FIELD OF THE PRESENT INVENTION

The present invention relates to ultrasonic devices and the various uses and methods thereof, and in particular to such devices for fertility control and management.

### BACKGROUND OF THE PRESENT INVENTION

A wide variety of contraceptive products exists on the market. The decision regarding the best method of birth control depends on various parameters such as overall health, age, frequency of sexual intercourse, sensitivity to material and drugs, subjective perception and sensation, etc.

The pill is frequently considered for birth control. Today the pill is available as combined oral contraceptives (COC) which contain both estrogen and progestin, or in progestin only pills (POP) which contain only progestin.

COCs work by suppressing ovulation and may make menstrual periods more regular. These pills are considered safe for most women, although women who smoke and are over 35 or who have a significant family history of cardiovascular disease should not use oral contraceptives due to an increased risk of cardiovascular disease. Women with a medical history of blood clots, or breast or endometrial cancers also should not use combined oral contraceptives. Possible side effects, which may subside after a few months, include nausea, headache, breast tenderness, weight gain, irregular bleeding, and depression.

POPs work by reducing and thickening the cervical mucus to prevent sperm from reaching the egg, and by keeping the uterine lining from thickening to prevent implantation of a fertilized egg. Because these pills contain no estrogen, the risk of blood clots is not present as with the combined oral contraceptives. This type of birth control pill is a good option for women who cannot take estrogen because they are breast feeding, or because of headaches or high blood pressure problems associated with estrogen, but the progestin only pill may cause menstrual changes, weight gain, and breast tenderness.

Injectable progestins also may used to prevent pregnancy up to three months by injection of Depo-Provera. Depo-Provera prevents pregnancy by inhibiting ovulation, changing the cervical mucus to prevent sperm from reaching an egg, and by changing the uterine lining so that a fertilized egg will be unable to implant. Benefits and side effects of Depo-Provera are similar to those of progestin only pills.

Interuterine Devices (IUD) are another type of contraceptive device. The IUD has experienced some bad publicity in the past when the Dalkon Shield was associated with a high incidence of pelvic infections, infertility, and some deaths. However, today's IUDs have been improved and have lower failure rates compared to most contraceptive methods. An IUD is a T-shaped device that is inserted into the uterus by a health care professional. There are two types of IUDs available, the Paragard CopperT 380A which protects against pregnancy for 10 years, and the Progestasert Progesterone T which must be replaced every year. The IUD is not suitable for the prevention of sexually transmitted diseases or infections.

Condoms also are used as contraceptives and to prevent transmission of sexually transmitted diseases. This device, usually made of latex, or more recently polyurethane, is used during sexual intercourse. It is put on a man's erect penis and physically blocks ejaculated semen from entering the body of a sexual partner. This is a barrier contraceptive that has a high failure rate compared to other products (over 10%) and it is also a physical barrier that harms the intimacy and pleasure of the sexual act.

An additional product is the sponge, which releases spermicidal gel over the vaginal mucus while the sponge forms a barrier to kill or immobilize sperm before they can reach the cervix and enter the uterus. The sponge can be inserted several hours before intercourse and can be left in place up to 12 hours after sex. Women who are allergic to nonoxyl-9 or who have had toxic shock syndrome should not use the sponge. The sponge failure rate is much higher than that of IUDs, for example.

A diaphragm is available by prescription and is sized by a health care professional to ensure a proper fit. The diaphragm works by covering the cervix with a dome-shaped rubber disk with a flexible rim to prevent sperm from entering the uterus. A spermicidal is applied to the diaphragm before insertion to kill sperm. Diaphragms should never be left in for more than 24 hours due to risk of toxic shock syndrome (TSS).

The cervical cap is similar to the diaphragm. It is a soft rubber cup with a rounded rim and is sized to fit, by a health care professional, tightly around the cervix. Like the diaphragm, spermicide is required with the cervical cap. It protects against pregnancy for 48 hours and for multiple acts of sexual intercourse during this time. Prolonged use (over 48 hours) may increase the risk of TSS and can produce a foul odor or discharge.

Vaginal spermicides are available over the counter in the form of cream, jelly, foam, film, vaginal suppository or tablets. These products contain a sperm killing chemical. There is debate about the effectiveness of using vaginal spermicides alone but it is thought that they have a failure rate of approximately 21% per year. In addition to the high failure rate, women who choose this method of contraceptive must follow the package directions carefully and exactly in order to not harm the effectiveness of this contraceptive, which also may be difficult to maintain.

Thus, the current contraceptive products on the market have major disadvantages such the need for active involvement of health car professional, side effects such as nausea, headache, breast tenderness, weight gain, irregular bleeding, and depression and even medical dangers such as TSS, pelvic infections, infertility, cardiovascular diseases, blood clots and even deaths. Additionally, the existing products do not have zero failure rates.

On the other hand, approximately 15% of couples attempting to conceive fail to do so within one year of unprotected intercourse. Fertility specialists define these couples as being infertile. 40% of these cases result from male factors and 60% from female factors. Treatment is available to ameliorate or relieve a condition which leads to infertility.

Some of these factors include the motility and concentration of the male sperm, or the viscosity level (“sticky” level) of the mucus in a woman's cervix. These factors influence both the probability and the duration for natural conception. In order to improve fertility and conception, the intervention of health car professional and drug therapy is required today, but this intervention involves procedures which do not lend themselves to home use, thus requiring implementation by a skilled practitioner.

Apart from the barrier method of a condom, male contraceptives have not generally been developed. Such a deficiency may at least in part relate to the complexity of the male reproductive tract and the difficulty of intervention therein. The following structures and origin secrete the substances that, together, make up the seminal fluid structure:
1) testes and epididymis—spermatozoa constitute less than 5% of the seminal fluid volume
2) Seminal vesicles—their secretions are reported to contribute about 60% of the seminal fluid volume.
3) Prostate gland—its secretions constitute about 31% of the seminal fluid volume.
4) Bulbourethral glands—their secretions are reported to contribute about 5% of the seminal fluid volume.

The spermatozoa need to travel the distance from their place of origin to the exterior. The sperm must pass from the testis through the epididymis, vas deferens, ejaculatory duct, and urethra.

There has long been interest in the medical community to be able to separate human male (Y chromosome containing) sperm cells and female (X chromosome containing) sperm cells in order to determine the sex of the offspring.

Furthermore, the separation of X and Y sperm cells also has great interest for animal breeding and production. The ability to separate X and Y sperm cells from each other, and to thereby have the ability to choose the gender of the progeny, is clearly advantageous in many agricultural industries. For example, in the bovine industry, cows are preferred for milk production while bulls are preferred for meat production. Many different techniques have been tried but there is no simple technique for separation of X- and Y-sperm cells. Moreover, currently available techniques and procedures require special tools such as centrifugation and microscopy, as well as technically skilled practitioners.

One of the techniques that was claimed to be at least qualitatively successful is immuno-magnetic separation and flow cytometry. Using this method, an enrichment of X or Y sperm cells of up to 98-99% was reported (A. T. Peter, P. P. Jones and J. P. Robinson, Theriogenolgy 11-1184, 1993; Sex pre-selection by DNA: Uptake on success of flow cytometric sperm sorting for shifting the sex ratio to 90: 10 or more. L. A. Johnson, J. R. Dobrinsky and G. R. Welch. Abstract P24-1, The 13th International Congress On Animal Reproduction, Sidney, Australia 1996). These techniques are technically very complicated and inefficient; furthermore, they provide too few sperm cells per time unit to make them suitable for routine use.

In U.S. Pat. No. 4,927,749 a colloidal density gradient medium is described which is the to enable separation between X- and Y-sperm cells by continuous density centrifugation in the medium for 0-15 minutes at 2.000× g and a start density of 1.185 g/mL, which is the to be between the densities of human X and Y sperm cells. In this technique, the sperm cells are not separated from debris and other possible contaminants, and the separation process most probably impairs the cell quality. More importantly, sperm cells separated according to U.S. Pat. No. 4,927,749 are likely to have very low viability and the product is therefore not very useful for purposes of conception.

### SUMMARY OF THE PRESENT INVENTION

There is an unmet need for, and it would be highly useful to have, a device that is able to use acoustic energy and force for management and control of fertility.

The present invention overcomes these drawbacks of the background invention by providing a device for fertility control and management through the application of acoustic energy, preferably ultrasound. According to various embodiments of the present invention, such fertility management and control may optionally be applied for reducing or enhancing fertility and/or otherwise controlling one or more aspects of fertility and conception, including but not limited to contraceptive use, improving the ability to conceive, and/or separation of X and Y sperm.

The device applies acoustic energy to the spermatozoa (also called sperm or spermatozoan) which comprise approximately 2-5% of the seminal fluid, in order to control and/or enhance the motility and movement of spermatozoa. Preferably the acoustic energy comprises ultrasound; more preferably the level and type of ultrasound, and the frequency or rate of application, are adjusted according to the desired outcome. For example, optionally the amount of applied ultrasound energy is higher for destroying, blocking and/or disabling sperm as described herein, and is lower for guiding and/or separating sperm, in order to avoid damage to the sperm. The ultrasound may optionally be applied continuously, in a single burst or as pulses, according to the requirements of the application and desired outcome.

Sound waves may be viewed as being generally mechanical in that they consist of the vibration of molecules about their equilibrium positions. Sound waves with frequencies above the upper limit that is audible to the human ear (about 18,000 Hz) lie in the ultrasonic range. There are two main classes of ultrasound presently in clinical or research use: (1) High frequency (4-7 MHZ), low-power ultrasound, which is employed extensively in diagnostic ultrasonography; and (2) Low-frequency (20 to 1000 kHz), high power ultrasound which has therapeutic uses.

It has been known for some time that the application of acoustic energy on a stream of flowing liquid will have an effect upon the behavior of gas bubbles entrained therein. An article entitled “Acoustic Effects on Gas Bubbles In the Flows of Viscous Fluids and Whole Blood”, which appeared in the Journal of Acoustical Society of America, 53, 5, 1327-1335, I. C. Maceto and Wen-Jeo Yang (1973), discussed the use of acoustic or ultrasonic waves to trap small bubbles against the wall of the tube in which the liquid stream is flowing, by using liquids that resemble whole blood in their Theological property. It was shown that the bubbles could be deflected and trapped against the sidewall of the tube in which flow is occurring. In 1992, Schwarz, Karl Q. et al., published an article entitled “The Acoustic Filter: An Ultrasonic Blood Filter for the Heart-Lung Machine”, in the Journal of Thoracic and Cardiovascular Surgery, 104, 6, 1647-1653 (December 1992). This article indicated that microbubbles in a chamber can be pushed to the opposite end of the chamber by using acoustic radiation energy.

U.S. Pat. No. 5,022,899, entitled Sonic Debubbler for Blood, hereby incorporated by reference as it fully set forth herein, discloses devices that employ an ultrasonic transducer to produce low power anisotropic sound waves at about the resonant frequencies of bubbles, to drive the bubbles in a specific direction. Such bubbles may then be ejected by being drawn out through a fluid outlet port or trapped in a disposable open cell bubble trap. Power levels are regulated so as to remain below a level which would cause hemolysis from cavitation.

Furthermore, it has been known for some time that the application of acoustic energy on biological surfaces such as skin generates micro-pores in the biological surfaces that enable the passage of different substances.

Such properties also unexpectedly render ultrasonic energy suitable for application to control of sperm. Each sperm is about 60 microns long and 8 microns wide, which enables acoustic energy to control, block and direct the motility of sperm. The male reproductive tract is accessible to ultrasound energy. As described herein in some embodiments, sperm may also optionally be controlled with acoustic energy outside of the male reproductive tract.

The present invention provides, in some embodiments, devices for controlling sperm motility and movement (blocking the movement of sperm in the male reproductive system during ejaculation or alternatively for enhancing sperm motility in the female reproductive system). In other embodiments, the present invention provides devices for separation of sperm from seminal fluid and/or further separation of Y sperm from X sperm.

In one aspect the present invention can be used as a contraceptive device and product, wherein the device uses acoustic energy, preferably ultrasonic energy, to cause spermatozoa traveling from the testes and epididymis to be blocked from reaching the exterior (ejaculation) in a manner that the ejaculated seminal fluid shall not contain the spermatozoa substance or alternatively contains dead sperm or at least a very low concentration of live sperm. This process divides the seminal fluid into first and second components. The first component is the seminal fluid component secretions without the spermatozoa; it is the component ejaculated and expelled outside. The second component, which is preferably blocked within the male reproductive system, contains the spermatozoa and other possible substances that have been removed or separated from the first component.

In one particular aspect, the present invention provides a contraceptive device for blocking sperm from the seminal fluid during ejaculation, which preferably comprises a transducer positioned in the vicinity of the male reproductive system or attached to the body or penis within which a seminal fluid is flowing during ejaculation, and a power source for operating the transducer to direct ultrasonic energy radially inward to the male reproductive system so as to block ejection of sperm in the seminal ejaculate material.

The spermatozoa constitute less only 2-5% of the seminal fluid volume. By blocking, withholding or destroying the spermatozoa in the male reproductive system using acoustic energy and not allowing the spermatozoa to reach the exterior with the rest of the seminal fluid during ejaculation, a more effective device can be constructed that also does not harm the intimacy and pleasure of the sexual act.

In another aspect, the present invention provides a device to enhance sperm motility in the female reproductive system and thus improve the probability of a female to conceive, preferably comprising a transducer for association with the exterior surface of the anterior side of the region of the urogenital triangle; and a power source for the transducer to generate ultrasonic waves that are directed toward the anterior side of the urogenital triangle and more preferably specifically toward the female vagina, cervical canal, body of uterus and the uterine tubes. Furthermore, the device may optionally be inserted into the vagina or adjacent to the vagina anterior to enhance the ability of the ultrasonic waves to direct the sperm into the uterine tube in which the egg is fertilized by a sperm.

According to some embodiments, the device is implemented as an apparatus for being attached and/or worn by a male or female, for example by being implemented as a device to be fitted on, in or near the penis or vagina, and/or as a device to be worn on a belt or other item of clothing, for example.

In another particular aspect, the present invention provides a device for separating sperm from the seminal fluid sample, comprising a seminal fluid sample container with inlet and outlet portion or other sterile withholding element, a transducer for association with the seminal fluid sample container, a power source drive circuitry (such as a pulse generator) for the transducer to generate ultrasonic waves that direct the sperm toward the collecting outlet in the sample container, and a collector for receiving the separated sperm from the collecting outlet.

In another aspect, the present invention provides separation of Y sperm and X sperm due to the differences in the size and motility of X and Y sperms, such that by applying acoustic force on a seminal sample, a gradient is generated during the movement of sperm from the inlet portion to the outlet portion. This would be particularly useful to improve the probability of a male or a female fetus or fetuses being conceived.

The device may optionally be incorporated and/or otherwise used with one or more existing contraceptives in order to combine the benefits of each technology. Optionally, the device may be integrated within other contraceptive products or medical treatment products, thereby producing a single unit comprising both components. Optionally each of the components may be differentially attached or coupled to one another, forming any number of combinations therefrom.

Optionally the device and other components may be attached or coupled thereto either during manufacture, or just before use.

In another aspect, this invention may also be used for animal breeding and production.

Hereinafter the term “X sperm” refers to sperm cells that contain the Y chromosome, while the term “Y sperm” refers to sperm cells that contain the X chromosome.

According to some embodiments of the present invention, there is provided a device for fertility control, comprising a source of acoustic energy, an acoustic transducer connected to the source of acoustic energy and a coupling element for coupling the acoustic energy to a fertility element.

Optionally the acoustic transducer and the coupling element are formed in a single element. Optionally, the fertility element comprises a penis and the coupling element comprises a flexible sleeve for being worn on or for encompassing the penis. Preferably the flexible sleeve comprises a condom. Also preferably, the acoustic transducer comprises a plurality of transducers disposed on the flexible sleeve. More preferably, the plurality of transducers is disposed on opposite sides of the flexible sleeve.

Optionally the coupling element is protected from contact with the seminal fluid by a biologically compatible barrier. Preferably the barrier is a thin film of flexible material.

Alternatively and more preferably, the plurality of transducers is disposed on the same side of the flexible sleeve.

Optionally the flexible sleeve further comprises an acoustic couplant. Preferably, the acoustic couplant comprises a wet couplant or a dry couplant. More preferably, the wet couplant comprises water or acoustic gel.

Optionally the acoustic energy is at a level for blocking, disabling or destroying sperm. Preferably, the flexible sleeve and/or couplant has a shape selected from the group consisting of cylindrical, concave and convex.

Optionally the fertility element comprises a penis and the coupling element comprises a belt for being worn near the penis. Preferably, the acoustic energy is at a level for blocking, destroying or disabling sperm.

Optionally the fertility element comprises a vagina and the coupling element comprises an element adapted for being placed in or near a female urogenitary region. Preferably, the element adapted for being placed in or near the urogenitary region comprises a pad and wherein the acoustic transducer is disposed on the pad. More preferably, a plurality of transducers is disposed on the pad for enhancing conception. Preferably, the element adapted for being placed in or near the urogenitary region comprises an element for being at least partially inserted to the vagina, the element comprising the acoustic transducer. This device is optionally adapted to enhance conception.

Optionally the element adapted for being placed in or near a female urogenitary region comprises a female condom.

Preferably the element is protected from contact with the vagina by biologically compatible barrier. More preferably, the barrier is a thin film of flexible material.

Optionally the fertility element comprises a sample container for the seminal sample external to a penis and collection container for collecting sperm wherein the coupling element couples the acoustic transducer to the sample container. Preferably, the container comprises an outlet placed at a location relative to a location of the acoustic transducer to receive motile X sperm or motile Y sperm or both.

Optionally the fertility element comprises a penis and wherein the coupling element comprises a flexible sample container for being placed over the penis, the device further comprising a harvesting port for collecting sperm from the container. Preferably, the harvesting port is placed at a location relative to a location of the acoustic transducer to receive motile X sperm or motile Y sperm or both.

Optionally the acoustic transducer comprises an ultrasonic transducer and the source of acoustic energy comprises an ultrasound source. Preferably, the device further comprises a controller for controlling the acoustic transducer.

According to other embodiments of the present invention, there is provided a method for separating X and Y sperm in a seminal sample, comprising: Applying acoustic energy to the seminal sample such that the sperm swim against or with the acoustic energy direction; and Separating the X and Y sperm according to relative location, wherein the X sperm location are more proximal in reference to the origin point and the Y sperm location are more distal in reference to the origin point.

Optionally the method further comprises separating motile and non-motile sperm. Preferably, the applying acoustic energy comprises collecting the sperm from the seminal sample in a container separated from the penis.

Optionally the applying acoustic energy comprises collecting the sperm from the seminal sample in a container worn on the penis during ejaculation to receive the sample directly.

Optionally the container comprises an impedance matched acoustic layer on the container interior walls to at least reduce acoustic wave reflection.

According to yet other embodiments of the present invention, there is provided a method for blocking conception, comprising applying acoustic energy to sperm in the female or male reproductive tract to block movement of sperm therein or to destroy or disable sperm therein.

According to still other embodiments of the present invention, there is provided a method for enhancing conception, comprising applying acoustic energy to sperm in the female reproductive tract to enhance movement of sperm therein.

According to yet other embodiments of the present invention, there is provided a method for managing fertility in a subject comprising driving at least one acoustic transducer according to one or more parameters selected from one or more of a desired wave pattern, intensity or range of frequencies.

Preferably the subject is a mammal. More preferably the subject is a human. Alternatively and more preferably, the subject is an agricultural animal. Optionally, the device is adapted to be portable.

Optionally the acoustic transducer comprises one or more additional auxiliary transducers.

Optionally the fertility element comprises a penis and the coupling element comprises a patch-like holder attached to the body in the vicinity of the reproduction system including the testis. Preferably, the acoustic energy is at a level for blocking, destroying or disabling sperm.

Optionally the fertility element comprises a penis and the coupling element comprises a spring-like holder that extends partially around the circumference of the penis. Preferably the acoustic energy is at a level for blocking, destroying or disabling sperm.

According to an optional embodiment of the present invention, there is provided an ultrasonic device, method and system for separating or isolating fluid components from a fluid, optionally bodily fluids, optionally follicular or ovarian fluids, optionally female or male reproductive fluids, optionally, ovarian fluids, optionally and preferably seminal fluids and most preferably blood. Most preferably, the ultrasonic device comprises at least one or more of the acoustic transducers described above. Most preferably ultrasonic fluid separation process is carried out in an extracorporeal or in vitro environment for example including but not limited to a reaction chamber, test tube, reaction surface, microtubule, microscopic slide, beaker, flask, vessel or the like in vitro environment. Optionally ultrasonic fluid separation process is carried out in an in vivo environment. Optionally and preferably, the ultrasonic device, method and system for separating a bodily fluid further provides for analyzing the isolated or separated fluid components preferably providing for fluid and fluid component analysis for example including but not limited to at least one or more of blood works analysis, urine analysis, seminal fluid analysis, sperm motility analysis, ovarian fluid analysis, follicular fluid analysis, lab work testing, pH analysis, hormone analysis, cellular analysis, oncogenic analysis, disease state analysis, immune system analysis or the like.

According to an optional embodiment of the present invention, there is provided an ultrasonic device, method and system for introducing, implanting, inserting, removing, placing, displacing, extracting, directing, navigating or otherwise controlling, the motility, movement or displacement of an entity associated with an in vivo environment toward a targeted location. Optionally, the controlled movement may be from an in vivo environment to an in vitro or extracorporeal environment. Optionally, the controlled movement may be from an in vitro or extracorporeal to an in vivo environment. Optionally, the controlled movement may be within an in vivo environment. Optionally, the movement control may be utilized for a plurality of optional entities within an in vivo environment for example including but not limited to nanodevices, nanoparticles, sensors, microsensors, small scale devices, or the like. Optionally and preferably, the ultrasonic device, method and system for movement control of entity within an in vivo environment may be applied to a plurality of optional in vivo environments or space for example includes but not limited to bodily fluid, blood, cellular environment, organ, tissue, skin, cardiovascular system, neural system, lymphatic system, ECM, immune system, auditory, visual, and the like in vivo environment.

For example, an optional depiction or application of the ultrasonic device providing movement control of an entity within a given in vivo environment or space is provided wherein a nanodevice to be removed and/or extracted form the cardiovascular system is moved and or directed within the cardiovascular system—using the ultrasound energy provided by the device system and method according to the present invention—toward a dedicated outlet for example including but not limited to a port, hypodermic needle, tube, angioplasty grabber, angioplasty catheter or the like tool bridging the in vivo environment with the external environment.

Unless otherwise defined, all technical and scientific terms used herein have the same meaning as commonly understood by one of ordinary skill in the art to which this invention belongs. The materials, methods, and examples provided herein are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 shows an exemplary, illustrative embodiment of a device according to the present invention for blocking, disabling and/or destroying sperm in the seminal fluid, including but not limited to seminal fluid discharged during ejaculation;

FIG. 2 shows an exemplary, illustrative non-limiting embodiment of a device according to the present invention for improving and enhancing the sperm motility in the female reproductive system and thus improving the probability of conception;

FIG. 3 shows an exemplary, illustrative non-limiting embodiment of another contraceptive device according to the present invention for being worn on the penis;

FIG. 4 is a schematic diagram of an exemplary, illustrative non-limiting embodiment of a device according to the present invention;

FIGS. 5 and 6 each show an exemplary, non-limiting, illustrative device according to the present invention for combining an existing off the shelf contraceptive product and the acoustic contraceptive device as described herein;

FIG. 7 shows another exemplary, illustrative implementation of a device according to the present invention for enhancing conception;

FIG. 8 illustrates an exemplary, non-limiting embodiment of the present invention in which one or more transducers are optionally placed on a belt;

FIG. 9 illustrates various exemplary implementations of a coupling element to be used between the transducer and the body for good energy flow and sterility;

FIG. 10 illustrates an exemplary, non-limiting device according to the present invention for separation of sperm from the seminal fluid; and

FIG. 11 illustrates an exemplary, illustrative device for separation of Y sperm from X sperm in the seminal fluid.

### DETAILED DESCRIPTION OF THE PREFERRED EMBODIMENTS

The present invention is of a device for fertility control and management through the application of acoustic energy, preferably ultrasound. According to various embodiments of the present invention, such fertility management and control may optionally be applied for reducing or enhancing fertility and/or otherwise controlling one or more aspects of fertility and conception, including but not limited to contraceptive use, improving the ability to conceive, and/or for separation of X and Y sperm.

The principles and operation of the present invention may be better understood with reference to the drawings and the accompanying description.

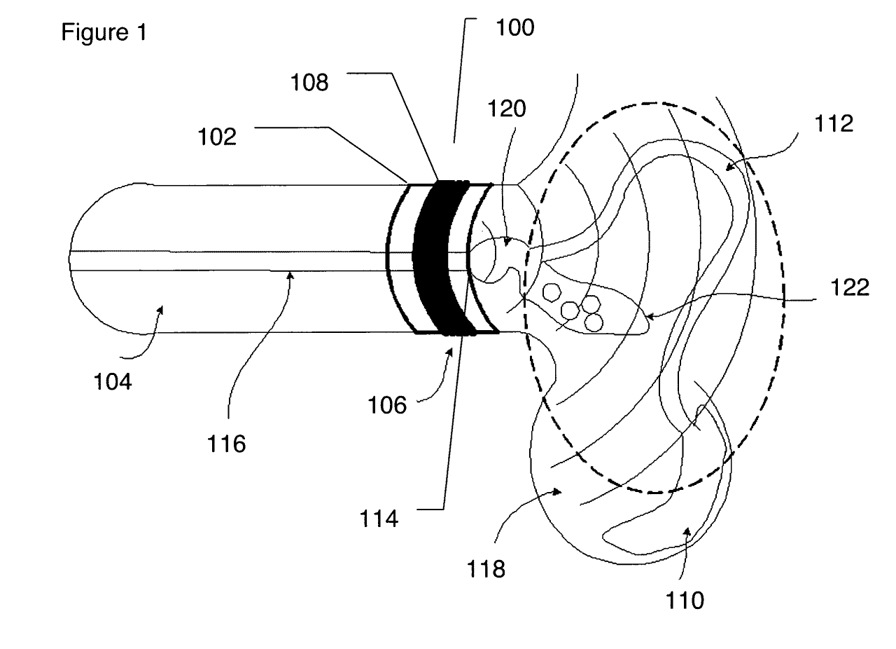

Referring now to the drawings, FIG. 1 shows an exemplary, illustrative embodiment of a device according to the present invention for blocking, disabling and/or destroying sperm in the seminal fluid, including but not limited to seminal fluid discharged during ejaculation.

As shown, a device 100 features a sleeve 102 for surrounding the proximal portion of the penis 104, which is preferably constructed of a flexible material, including but not limited to one or more of rubber, latex, polyurethane and the like. Sleeve 102 features an acoustic device for emitting acoustic energy, such as for example an acoustic transducer 106 as shown (as a non-limiting example only). Acoustic transducer 106 preferably has an annular shape as shown, although other shapes are possible. Acoustic transducer 106 preferably comprises at least one and more preferably a plurality of piezo crystals 108. Acoustic transducer 106 may also optionally comprise any piezoelectric, magnetrostrictive, or other form of transducer that produces ultrasonic waves. Sleeve 102 preferably acts not only as a support structure but also as a coupling element to minimize the space between the acoustic transducer 106 and the tubular body of the penis 104.

Optionally, sleeve 102 comprises an acoustic gel or any other material with a good flow path for acoustic energy therethrough. Sleeve 102 may optionally be made from and/or filled with such acoustic material, which are also referred to as “acoustic couplants”. Non-limiting examples of such is acoustic couplants include water, oil, glycerin, and various gels suitable for ultrasound sonagraphy as wet couplants. Dry couplants optionally include but are not limited to rubber, elastomeric materials and alloys of indium and gallium. Combinations of such acoustic materials may also optionally be used.

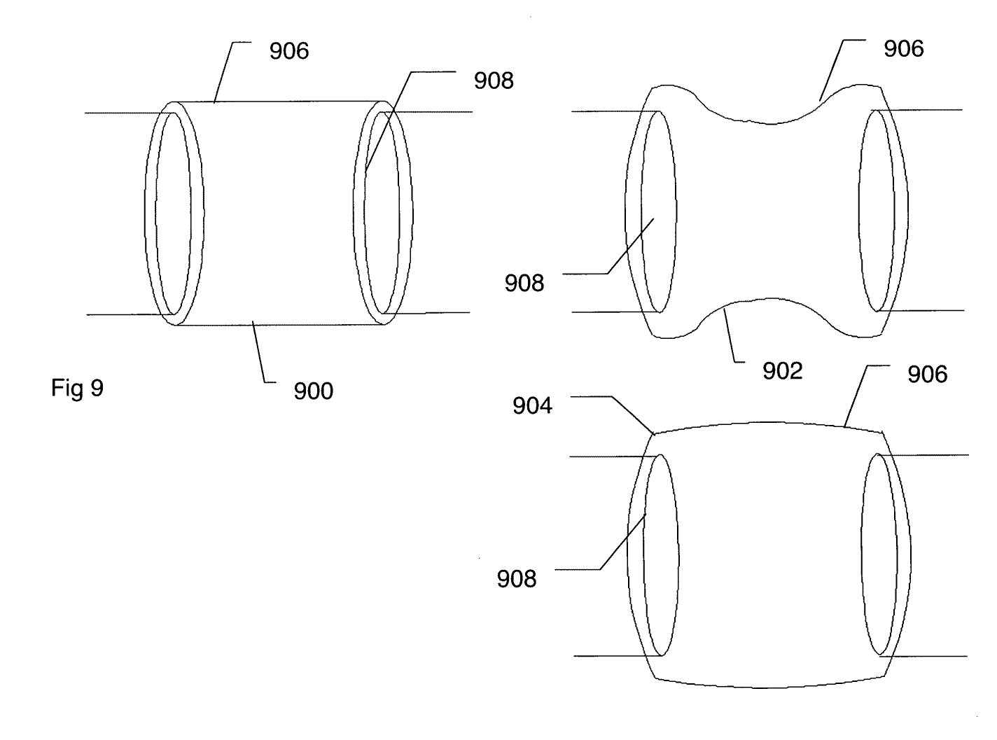

Optionally, to further improve acoustic energy flow, an add-on or integral coupling element may optionally be attached or incorporated in sleeve 102 and interspaced between acoustic transducer 106 and the penis 104 (not shown; see FIG. 9). The coupling element may optionally be filled with any of the above acoustic couplants so that there will be a good flow path for acoustic energy therethrough.

In place of sleeve 102, a spring-like holder that extends only partially of the circumference of the penis 104 may optionally be used (for example the holder may extend for about 210° of the circumference; not shown). The holder preferably positions the piezo crystals 108 at the desired location on the circumference. Thus, the holder will generally minimize the associated area with the male penis 104.

Furthermore, in place of sleeve 102, a patch-like holder attached to the body in the vicinity of the reproduction system including the testis 118 may also optionally be used (not shown). The holder preferably positions the piezo crystals 108 at the desired location to apply acoustic waves in the desired location and direction.

With regard to the embodiment shown, before ejaculation, the sperm in the seminal fluid stream passes through and travel the distance from their place of origin to the exterior. The sperm must pass from the testis through the epididymis 110, vas deferens 112, ejaculatory duct 114, and urethra 116 as shown. During this emission process, preferably acoustic energy is applied to the sperm from acoustic transducer 106, as the elapsed time period required for the seminal fluid emission process is sufficient to physically apply force on the spermatozoa and block their movement inside the male reproductive system. Acoustic transducer 106 is operated so as to generate ultrasonic waves and direct them radially inward towards the reproduction system. Accordingly, sperm are blocked, disabled or destroyed and the ejaculated seminal fluid most preferably either does not contain sperm or alternatively contains dead sperm or at least a very low concentration of live sperm.

The sperm is blocked during emission by acoustic waves that apply net force on the sperm in a direction which is opposite to the seminal fluid emission path, thereby maintaining the sperm within the male reproductive system. The process of disabling or destroying sperm occurs when acoustic waves interfere with the process of “ripening”, or maturation, as the sperm pass through the genital ducts before ejaculation and/or by impairing the sperm structure, motility or the ability of the sperm to undergo a capacitation process and timing which is very important in order to fertilize the ovum. For example, by impairing the sperm acrosomal (head) cap which contains and releases hydrolytic enzymes during capacitation, the sperm is not able to break down cervical mucus or penetrate the outer covering of the egg and initiate fertilization. To maximize the effect of the acoustic waves, the transducer is preferably operated at frequencies corresponding to the resonant frequencies of the sperm.

In order to effectively block sperm regardless of its position/orientation in reference to the direction of the acoustic waves reaching the sperm (the range of sperm exposure size to acoustic waves is approximately 5 to 60 microns), the sound wave generated by the transducer optionally may combine a number of wavelengths to encompass the resonant frequencies of sperm sizes and sperm body exposure angle to the acoustic waves, while still being of sufficiently low power to avoid adverse effects on the body tissues and blood. In order to accomplish this, the transducer 106 is tuned to provide a broad band ultrasonic signal. Suitable signals to cause generation of low frequency and/or high frequency ultrasound waves may optionally be transmitted simultaneously by activating a pulse generator (not shown) connected to the various piezo crystals 108.

Alternatively, it may also be satisfactory to use the same crystals 108 to alternately produce ultrasound of the desired different frequencies. The acoustic frequency, and thus wavelength, can be varied in time over the desired wavelength range to effectively block sperm according to the variety of sperm sizes and exposure cross-section.

Different frequencies of sound waves are optionally generated during operation of device 100.

Ultrasound energy is also preferably applied to the testis 118 as shown for additionally increasing the efficacy of device 100. Ultrasound energy as applied to the epididymis 110, vas deferens 112, ejaculatory duct 114, prostate gland 120 and/or the seminal vesicle 122 as shown is sufficiently low in intensity to not heat or damage body but is sufficiently strong and directional to physically apply force on the spermatozoa and prevent them from reaching the exterior during ejaculation.

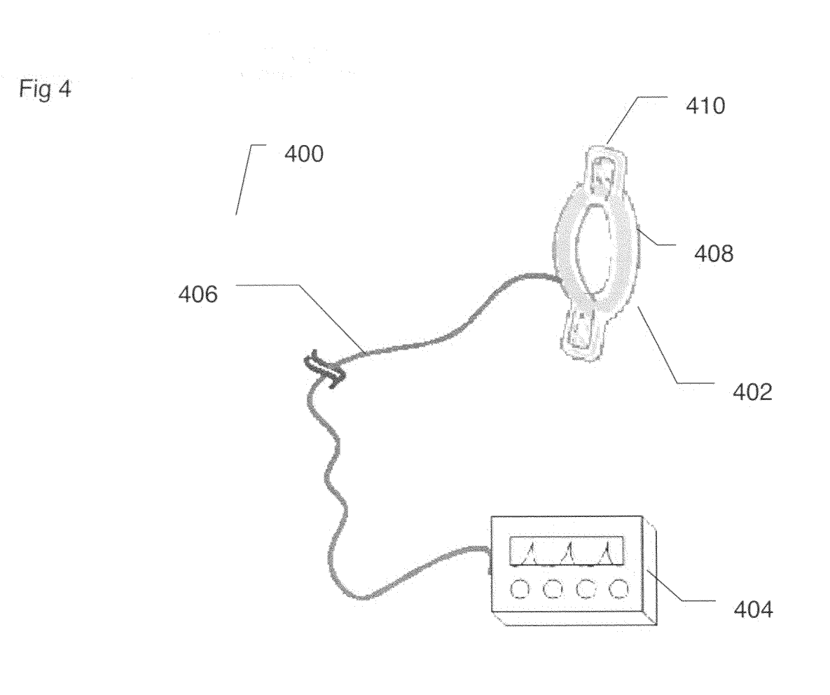

It should be noted that the device illustrated in FIG. 1 does not show the device power generator which may be optionally incorporated within device 100 or more preferably connected to device 100 externally as described in FIG. 4 for example.

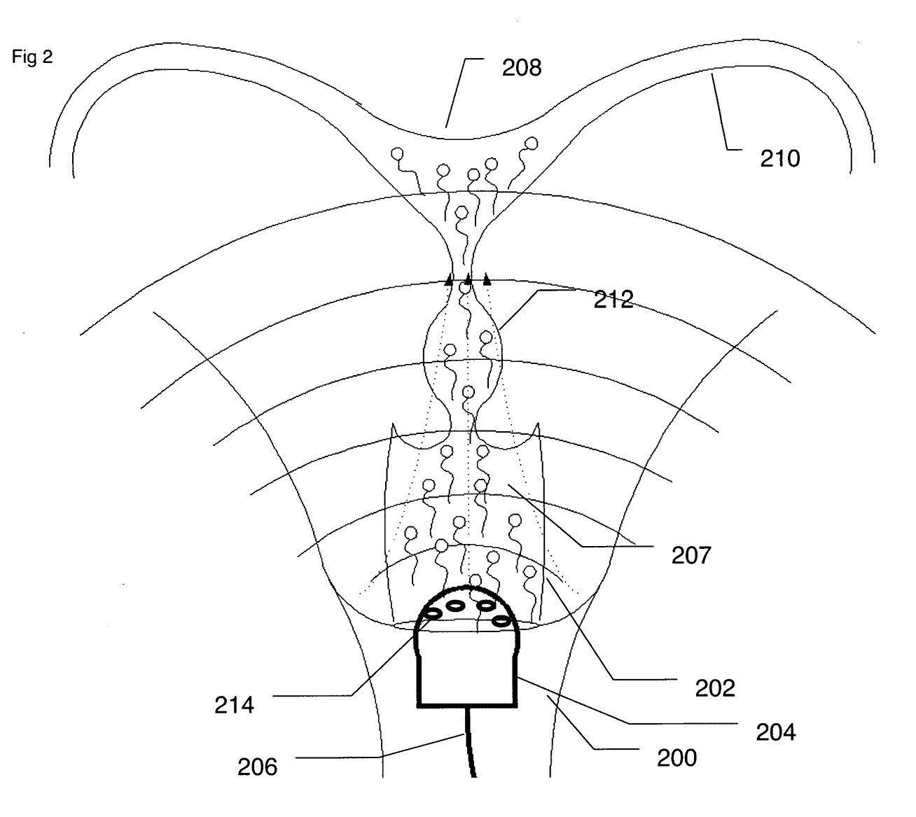

FIG. 2 shows an exemplary, illustrative non-limiting embodiment of a device according to the present invention for improving and enhancing the sperm motility in the female reproductive system and thus improving the probability of conception. A device 200 is optionally coupled to the exterior surface in the general region of the urogenital triangle (not shown) or alternatively is at least partially inserted into the vagina 202 as shown. Device 200 preferably features an acoustic transducer 204 coupled to an acoustic energy source (not shown) through a connector 206. After ejaculation, the sperm 207 in the seminal fluid stream travel the distance from their place of origin to the uterine body cavity 208 and uterine tube 210. During this period of time the sperm 207 may receive acoustic energy to generate a desired effect. Acoustic transducer 204 is preferably operated so as to generate ultrasonic waves and direct the sperm 207 radially inward towards the female reproduction system, such that the ultrasonic waves are applied as the sperm travel through the vagina 202, the cervical canal 212 and the uterine body cavity 208. Accordingly, sperm will be provided with a force that increases the motility of the sperm inwardly. Furthermore, it has been known for some time that the application of acoustic energy on biological surfaces such as skin generates micro-pores in the biological surfaces that enable the passage of different substances. Thus, the ultrasonic waves may be expected to both provide force to increase the motility of the sperm inwardly, and also to generate a path for sperm entry by generating micro-pores in the cervical mucus and the outer covering of the egg to initiate fertilization.

Acoustic transducer 204 preferably features at least one and more preferably a plurality of piezo crystals 214 arranged as an array, such that the crystal(s) enter to vagina 202 as shown. Piezo crystals 214 are preferably focused in a plurality of different directions, more preferably inward towards the cervical canal 212, body of uterus 208 and the uterine tubes 210 along the path of the sperm. Acoustic transducer 204 may also optionally comprise any piezoelectric, magnetrostrictive, or other form of transducers that produces ultrasonic waves.

Suitable signals to cause generation of high frequency and/or low frequency ultrasound waves may optionally be transmitted simultaneously by activating a pulse generator (not shown) connected to the various piezo crystals 214. The employment of a plurality of crystals 214 is preferred so that one or more crystals 214 may optionally be dedicated to providing high frequency sound waves; however, it may also be satisfactory to use the same crystals 214 to alternately produce ultrasound of the two different desired frequencies. Low, high and mixed frequency sound waves are optionally generated during operation of device 202.

When the transducer 204 is at least partially inserted in vagina, it is preferred that transducer 204 is protected from contact with the vagina 202 and vagina fluids by a thin film (not shown) of flexible material interspaced between the vagina 202, vaginal fluids and the transducer 204. This thin film permits passage of the ultrasonic waves while preventing vaginal fluids from damaging the transducer 204; such a cover also prevents transducer 204 from contaminating the vaginal fluids and seminal fluid. Various plastic or polymeric films which are biologically compatible are useful for forming this barrier.

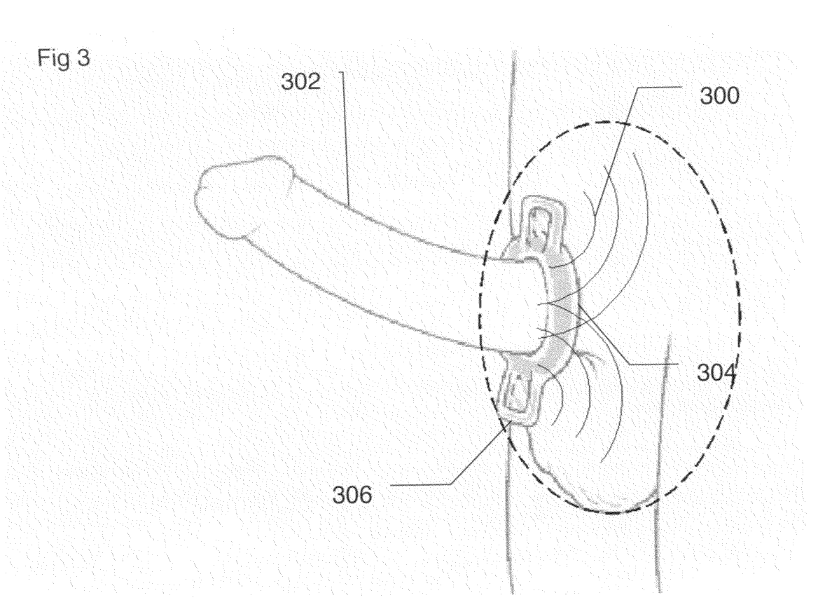

FIG. 3 shows an exemplary, illustrative non-limiting embodiment of another contraceptive device according to the present invention for being worn on the penis. A device 300 comprises a sleeve 304 for being worn on the penis 302. Sleeve 304 preferably features a plurality of transducers 306, of which two are shown for the purpose of illustration only and without any intention of being limiting. Optionally only one transducer 306 is used (not shown). Transducers 306 may optionally be square, round, shaped in a ring and/or incorporated within sleeve 304, and/or may feature any other suitable shape. Transducers 306 preferably generate ultrasonic waves simultaneously or separately, for example sequentially. The waves are preferably directed inwardly to the reproduction system.

The device 300 may optionally generate high frequency and/or low frequency ultrasound waves to be transmitted simultaneously by activating a pulse generator connected to the various piezo crystals (not shown). The employment of a plurality of crystals is preferred so that some may be dedicated to providing high frequency sound waves; however, it may also be satisfactory to use the same crystals to alternately produce ultrasound of a plurality of different desired frequencies. Low, high and mixed frequency sound waves are optionally generated during the operation of device 300.

To further improve the reliability of the device 300, optionally some of the transducers or an additional transducer (not shown) may be used as auxiliary transducers. This auxiliary transducer may operate together with the primary transducer or be activated automatically upon primary transducer failure as a back-up. Thus, the auxiliary transducer provides a second level of defense to guard against sperm reaching the exterior during ejaculation.

Because the device 300 may need to be operated for a lengthy period of time during sexual intercourse, the device 300 preferably contains a set of transducers 306 that are adjacent to each other or on both sides of the penis 302, as shown. In order to avoid potential adverse side effects upon the tissues from these ultrasonic waves, the device 300 may be operated to alternately send signals first to the set on one side of the penis 302 for a first period, such as a few minutes for example, and then to the set on the other side of the penis 302 for a second period, to generate the desired wave patterns in the regions of the male reproductive system without substantially heating or otherwise affecting the tissues.

FIG. 4 is a schematic diagram of an exemplary, illustrative non-limiting embodiment of a device according to the present invention. As shown, a device 400 features an acoustic transducer 402 and a power source and pulse generator 404 for operating acoustic transducer 402. Power source and pulse generator 404 preferably provides power through a connector 406. Acoustic transducer 402 preferably features a sleeve 408 comprising piezo crystals (not shown) with a plurality of transducers 410 as shown. Alternatively acoustic transducer 402 may comprise a spring-like holder that extends for about 210° of the circumference of the penis and positions the piezo crystals at the desired posterior location (not shown).

Acoustic transducer 402 may optionally be activated by a power source and pulse generator 404, which is connected to the various piezo crystals. Power source and pulse generator 404 also preferably enables device activation and deactivation, provides one or more device indications and controls transmitted power to each transducer 410.

A disposable element may optionally be placed between the transducer 410 and the body (not shown). The disposable element is preferably sterile and may optionally be filled with water, a gel or any other suitable acoustic couplant as described herein. Optionally the coupling element could be reused by employing materials that can be sterilized after use.

Preferably, the excess weight of the portion associated with the body is minimized, as is the total weight of the device 400 itself. Any components which do not need to be placed in contact with the body and/or very close to the body are most preferably incorporated in the power source and pulse generator 404. Most preferably the device 400 is portable and compact in size and weight.

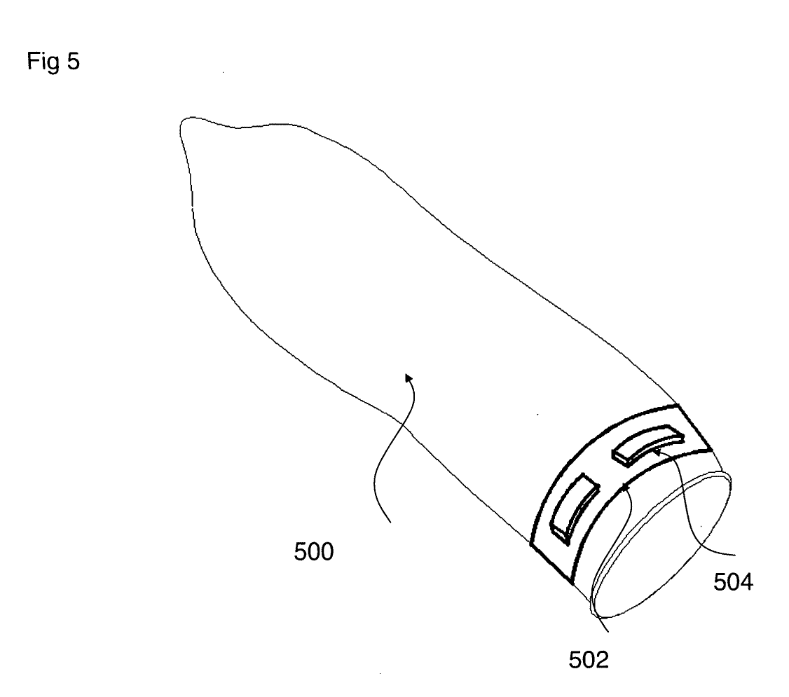

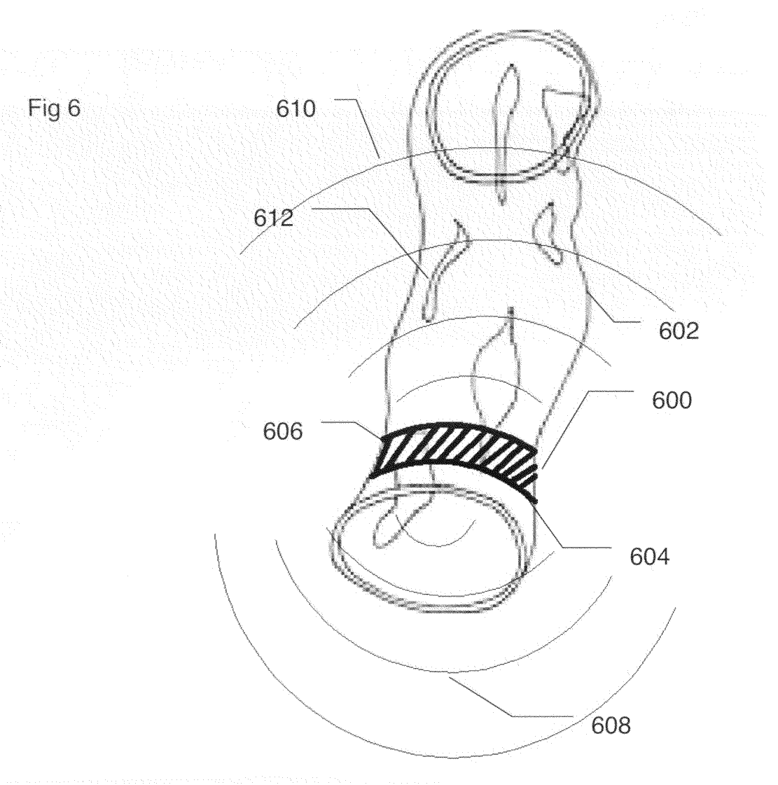

In some embodiments, the device according to the present invention may optionally be combined with another birth control method in order to reach a combined very low failure rate and optionally also to prevent the spread of sexually transmitted diseases such as HIV. FIGS. 5 and 6 each show an exemplary, non-limiting, illustrative device according to the present invention for combining an existing contraceptive product and the acoustic contraceptive device as described herein, for greatly improving over all safety.

As shown in FIG. 5, a condom 500 preferably features an add-on sleeve 502 with at least one transducer 504, of which a plurality is shown for the purpose of illustration only and without any intention of being limiting. Sleeve 502 and transducer 504 may optionally be implemented according to any of the previously described embodiments for a contraceptive device to be worn on the penis. An array of piezoelectric crystals, which is most preferably located in the proximal portion of condom 500 (to the interior), directs acoustic waves inward to the male reproductive system for blocking and disabling sperm before and during ejaculation. Optionally, the transducer 504 also produces acoustic waves directed outward to the ejaculated seminal fluid for destroying the remaining live sperm after ejaculation if any remain.

FIG. 6 shows a combination of an acoustic contraceptive device 600 according to the present invention with a female condom 602 to achieve a combined very low failure rate. Device 600 preferably features a sleeve 604 with at least one and more preferably a plurality of transducers 606. During intercourse, acoustic energy from transducers 606 interacts with the male penis (not shown) penetrating the female condom 602. Transducers are operated so as to generate ultrasonic waves 608 and direct them radially inward towards the penis. Accordingly, sperm will be blocked, disabled or destroyed and the ejaculated seminal fluid most preferably does not contain sperm or alternatively contain dead sperm or at least a very low concentration of live sperm. Optionally the transducer may also generate ultrasonic waves and direct them radially outward 610 towards the ejaculated sperm 612 in order to further destroy any remaining concentration of live sperm. Device 600 may optionally be implemented according to any of the embodiments for a male contraceptive device as described herein.

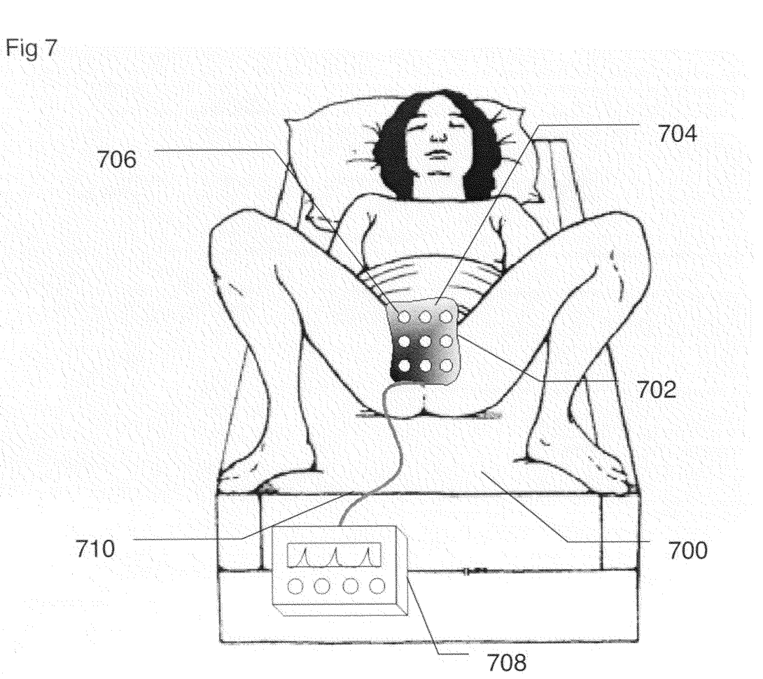

FIG. 7 shows another exemplary, illustrative implementation of a device according to the present invention for enhancing conception. Device 700 is an ultrasonic device which focuses on the urogenital triangle 702 of the female to enhance the sperm motility and direct the sperm to advance towards the cervical canal, body of uterus and the uterine tubes for improving the probability of conception. The device 700 preferably includes a relatively flat pad 704 of a suitable size and shape, which may optionally be attached to the urogenital triangle in a similar manner to that in which ECG electrodes are attached. The pad 704 preferably features at least one and more preferably a plurality of transducers 706 which are operated so as to create ultrasonic waves that move vertically inward through the vagina, cervical canal, body of uterus and the uterine tubes and thus preferentially cause sperm to move more easily and in larger quantities towards the uterus.

Transducers 706 are preferably comprised of at least one and more preferably a plurality of piezo crystals or any piezoelectric, magnetrostrictive, or other form of transducers that produces ultrasonic waves. Transducers 706 are preferably activated by a pulse generator or any drive circuitry. The pulse generator is preferably incorporated in a central controller 708 that also preferably enables device activation, provides one or more device indications, serves as power source and controls transmitted power to each transducer 706. Controller 708 is preferably connected to pad 704 through a connector 710.

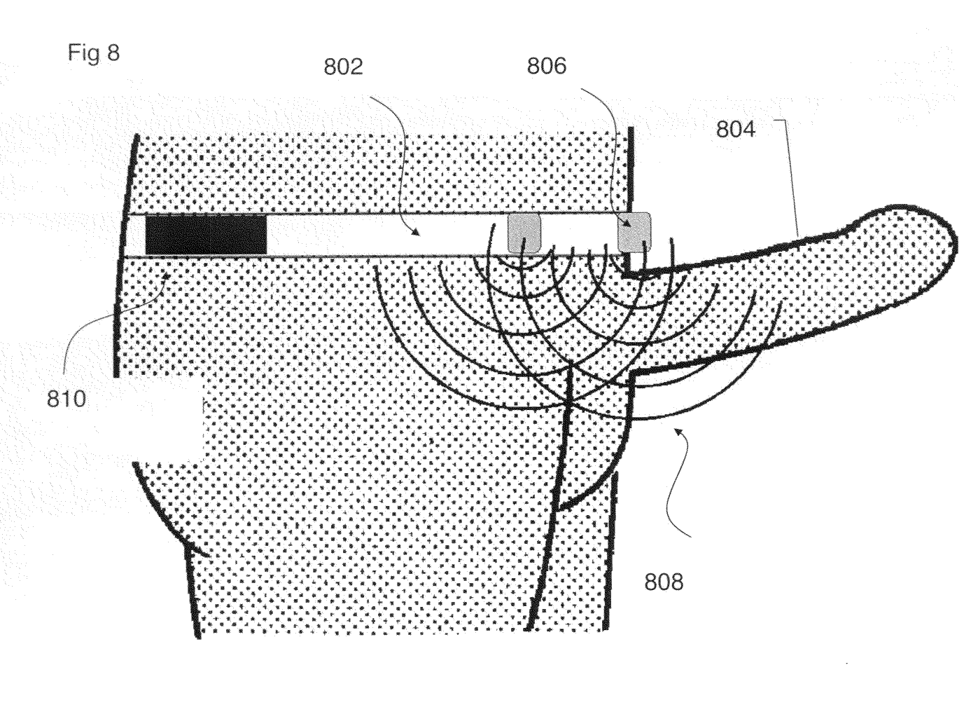

FIG. 8 illustrates an exemplary, non-limiting embodiment of the present invention in which one or more transducers are optionally placed on a belt. As shown, a device 800 preferably features a belt 802 for being worn at the abdomen of the male user, preferably close to the penis 804. The belt 802 preferably features at least one and more preferably a plurality of transducers 806, each of which preferably features one or more piezo crystals. Transducers 806 are preferably operated so as to create ultrasonic waves 808 and direct them radially inward towards the male reproductive system as previously described.

Optionally the pulse generator and control box 810 may be mounted on the belt 802 as shown, such that coupling is preferably provided through belt 802 using an isolated connector (not shown).

FIG. 9 illustrates various exemplary implementations of a coupling element to be used between the transducer and the body for good energy flow and sterility. A plurality of different acoustic elements 900, 902 and 904 are shown. Element 900 is preferably essentially annular or cylindrical; element 902 is preferably a distorted, concave annulus or cylinder; and element 904 is preferably a distorted, convex annulus or cylinder. The sterile elements 900, 902 and 904 may optionally be filled with water or with a gel, and/or any other acoustic couplant as described herein, so that there will be a good flow path for acoustic energy therethrough. Optionally the coupling element 900, 902 or 904 is made of disposable materials and/or alternatively of materials that can be sterilized after use.

Each element 900, 902 or 904 has an external surface 906 to be associated with the transmitting portion of the device (not shown) and an internal surface 908 to be associated with the body and/or another contraceptive device or product (not shown).

Optionally, each element 900, 902 or 904 may be constructed of materials that are suitable for being placed on the penis. For this implementation, the user can roll over or put the element 900, 902 or 904 on the erect penis or the transmitting portion of the device (not shown) to improve the flow of acoustic energy.

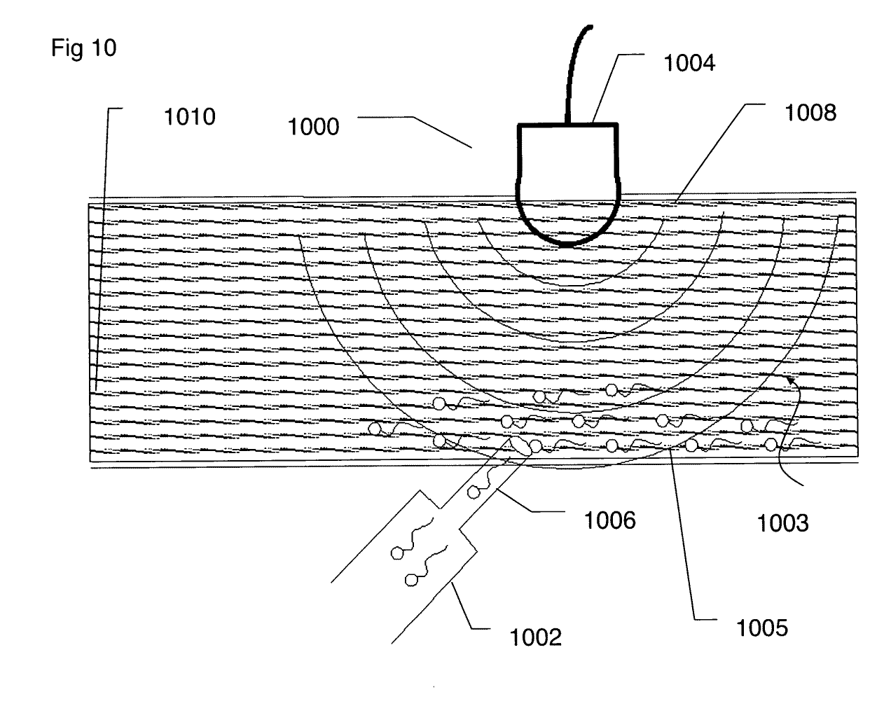

FIG. 10 illustrates an exemplary, non-limiting device according to the present invention for separation of sperm from the seminal fluid. The device 1000 features an sample container 1008 having an interior 1010, a container portion 1002 for collecting and housing the sperm, which may optionally contain a nutritional and/or preservative material to preserve the seminal fluid. Device 1000 also features a transducer 1004 for generating the acoustic waves 1003 to direct over time the sperm 1005 from the sample container 1008 to the container portion 1002. Container portion 1002 may optionally feature a collecting tube 1006 as shown.

The device 1000 also preferably features a more preferably elastic sample element 1008 having an interior 1010 which receives a penis (not shown) and receives directly the ejaculated seminal fluid with minimum contaminations. The interior 1010 may optionally contain a nutritional and/or preservative material to preserve the seminal fluid. The elastic sample element 1008 having an interior 1010 is preferably in fluid communication with container portion 1002 and in acoustic communication with transducer 1004.

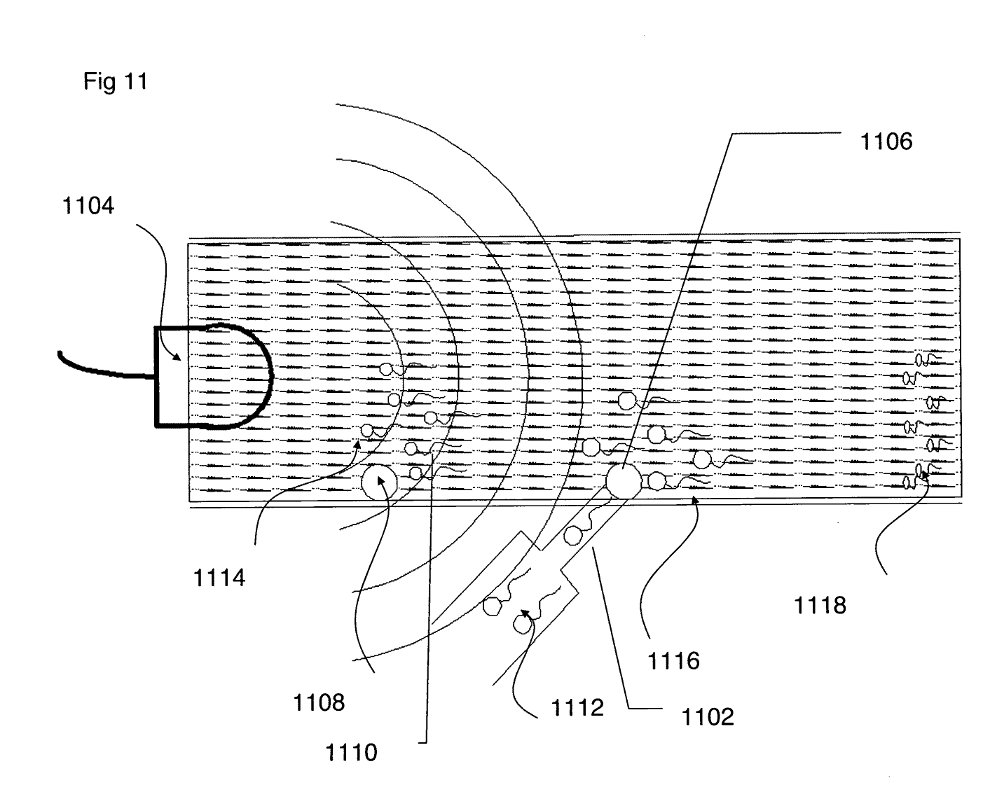

FIG. 11 illustrates an exemplary, illustrative device for separation of Y sperm from X sperm in the seminal fluid. The female sperm motility is lower as the X sperm are bigger than the Y sperm, thus upon applying acoustic force, a gradient is formed over time where the X sperm are more concentrated near the origin and the Y sperm are concentrated at a greater distance than the X sperm in reference to the starting point of the sample in the separation medium. The device 1100 for separating male sperm (Y) from female sperm (X) in the seminal fluid comprises a separation vessel 1102 and a transducer 1104 as shown.

Separation vessel 1102 includes an inlet port 1106, an outlet port 1108, and a separation medium (not shown) into which motile spermatozoa in the sample can flow via the inlet port 1106. The spermatozoa collecting inlet port 1106 is preferably an opening, hole or aperture in a wall 1110 of the device 1100, which may continue into a pipe, tube or conduit for harvesting the separated sperm, shown as a harvest tube 1112. Spermatozoa thus have to swim against acoustic force in order to travel from one end of the separation medium to the other end of the separation medium, thereby enhancing the separation of male sperm 1114 from female sperm 1116 and the separation of both male and female sperm 1114 and 1116 (motile sperm) from non-motile sperm 1118 which concentrate at the starting point of the separation medium.

It should be noted that spermatozoa separation from the seminal fluid may also optionally be performed when the acoustic force of transducer 1104 is directed to increase the motility of the sperm (i.e. the movement of the spermatozoa is supported by the acoustic energy, rather than being opposed by such energy). A gradient is still formed between the X sperm and the Y in the separation medium, which enables the separation of Y sperm from X sperm in the seminal fluid.

The reflection of acoustic waves is preferably minimized to prevent disruption of the acoustic wave fronts; otherwise the sperm movement and separation efficiency may be less than optimal. Such minimization may optionally be performed in various ways, for example by shaping the separation vessel 1102 to avoid reflective surfaces that might change the path of the acoustic waves. Another possibility is to use impedance matched acoustic material (not shown) on the inside of the walls of separation vessel 1102 walls to eliminate acoustic wave reflection from such surfaces. Many other high acoustic impedance materials are known in the art and can be used. The material layer is approximately a quarter wavelength thick at the frequency of the acoustic wave generated by the transducer. Thus, acoustic waves impinging on the inner walls of separation vessel 1102, and reflected back from the inner walls of separation vessel 1102, are one-half of a wavelength out of phase with the wave front at the inner surface of the walls, which causes cancellation of acoustic waves at the surface of the inner walls. It should be noted that if implementing the matching acoustic material, it diminishes to some extent the acoustic wave intensity.

The separation medium allows spermatozoa to migrate therethrough, in preference to non-motile sperm, which is preferred for artificial insemination of the female and/or for performing conception outside the female body. This can be achieved using any suitable buffer (e.g. HEPES, EBSS and the like), as motile spermatozoa will be able to migrate through the medium actively whereas, over a relevant time-scale, non-motile spermatozoa will at best enter passively by diffusion. It is preferred, however, to use a medium that enhances migration of motile spermatozoa from the sample. Suitable media include cervical mucus components [e.g. Keel & Webster (1988) Fertil. Steril. 49:138 143], polyacrylamide gel [e.g. Lorton et al. (1981) Fertil. Steril. 35:222 225], hyaluronic acid [e.g. Aitken et al. (1992) J. Androl. 13:44 54], or a cellulose derivative [e.g. international patent application PCT/GB00/03130 (Genosis Limited)] such as methylcellulose. The medium may be in the form of a solution or a gel.

For the above embodiments for separating sperm, preferably separation may also optionally be performed to separate sperm from other elements of seminal fluid which may contribute to infertility. Elements that are of the size of 4-100 micron and are not entirely liquid substance are possible to separate using the correct acoustic wave frequencies and intensity. Thus, it may be desirable to remove such materials in some cases from the sample.

While the invention has been described with respect to a limited number of embodiments, it will be appreciated that many variations, modifications and other applications of the invention may be made.

What is claimed is:

1. A device for fertility control, comprising a source of acoustic energy, an acoustic transducer connected to said source of acoustic energy and a coupling element for coupling said acoustic energy to a fertility element.

2. The device of claim 1, wherein said acoustic transducer and said coupling element are formed in a single element.

3. The device of claim 1, wherein said acoustic transducer comprises a plurality of transducers disposed on said flexible sleeve.

4. The device of claim 3, wherein said plurality of transducers is disposed on a side chosen from the group consisting of opposite sides of said flexible sleeve and the same side of said flexible sleeve.

5. The device of claim 1, wherein said flexible sleeve further comprises an acoustic couplant.

6. The device of claim 1, wherein said acoustic energy is at a level for blocking, disabling or destroying sperm.

7. The device of claim 1, wherein said fertility element comprises a penis and said coupling element comprises a belt for being worn near said penis.

8. The device of claim 7, wherein said acoustic energy is at a level for blocking, destroying or disabling sperm.

9. The device of claim 1, wherein said fertility element comprises a vagina and said coupling element comprises an element adapted for being placed in or near a female urogenitary region.

10. The device of claim 9, wherein said element adapted for being placed in or near said urogenitary region comprises a pad and wherein said acoustic transducer is disposed on said pad.

11. The device of claim 10, wherein a plurality of transducers is disposed on said pad for enhancing conception.

12. The device of claim 9, wherein said element adapted for being placed in or near said urogenitary region comprises an element for being at least partially inserted to said vagina, said element comprising said acoustic transducer.

13. The device of claim 12, adapted to enhance conception.

14. The device of claim 1, wherein said fertility element comprises a sample container for the seminal sample external to a penis and collection container for collecting sperm wherein said coupling element couples said acoustic transducer to said sample container.

15. The device of claim 14, wherein said container comprises an outlet placed at a location relative to a location of said acoustic transducer to receive motile X sperm or motile Y sperm or both.

16. A method for separating X and Y sperm in a seminal sample, comprising:
Applying acoustic energy to said seminal sample such that the sperm swim against or with said acoustic energy direction; and
Separating the X and Y sperm according to relative location, wherein the X sperm location are more proximal in reference to the origin point and the Y sperm location are more distal in reference to the origin point.

17. The method of claim 16, further comprising separating motile and non-motile sperm.

18. The method of claim 16, wherein said applying acoustic energy comprises collecting the sperm from the seminal sample in a container separated from the penis.

19. The method of claim 16, wherein said applying acoustic energy comprises collecting the sperm from the seminal sample in a container worn on the penis during ejaculation to receive the sample directly.

20. A method for blocking conception, comprising applying acoustic energy to sperm in the female or male reproductive tract to block movement of sperm therein or to destroy or disable sperm therein.

21. A method for enhancing conception, comprising applying acoustic energy to sperm in the female reproductive tract to enhance movement of sperm therein.

22. A method for managing fertility in a subject comprising driving at least one acoustic transducer according to one or more parameters selected from one or more of a desired wave pattern, intensity or range of frequencies.

23. The method of claim 22, wherein the subject is chosen from the group consisting of a mammal, a human and an agricultural animal.

24. The device of claim 1, adapted to be portable.

25. The device of claim 1, wherein said fertility element comprises a penis and said coupling element comprises a patch-like holder attached to the body in the vicinity of said reproduction system including the testis.

26. An ultrasonic device for introducing, implanting, inserting, removing, placing, displacing, extracting, directing, navigating a small scale entity associated with an in vivo environment.
